(19) United States
(12) Patent Application Publication
(10) Pub. No.: US 2015/0141820 A1
(43) Pub. Date: May 21, 2015

(54) ULTRASONIC GEL SHEET ASSEMBLY

(71) Applicant: SEIKO EPSON CORPORATION, Tokyo (JP)

(72) Inventors: Masayoshi YAMADA, Chino (JP); Kanechika KIYOSE, Matsumoto (JP)

(21) Appl. No.: 14/541,556

(22) Filed: Nov. 14, 2014

(30) Foreign Application Priority Data
Nov. 21, 2013 (JP) 2013-240698

(51) Int. Cl. A61B 8/00

(52) U.S. Cl. CPC A61B 8/4281
USPC 600/437

(57) ABSTRACT

The ultrasonic gel sheet assembly is used for acoustic matching between an ultrasonic device and an object to be tested. The ultrasonic gel sheet assembly includes a plurality of gel layers placed on top of each other. A partition sheet is placed between any adjacent ones of the gel layers. The partition sheet partitions a dispersion medium contained in the gel layers between the gel layers. Movement of water or any other dispersion medium is blocked by the partition sheet 14 between the adjacent gel layers.

### BACKGROUND

### Technical Field

The present invention relates to an ultrasonic gel sheet assembly and the like used for acoustic matching between an ultrasonic device and an object to be tested.

Ultrasonic imaging apparatuses such as an ultrasonic diagnosis apparatus are generally known. An ultrasonic device is incorporated in an ultrasonic prove of such an ultrasonic diagnosis apparatus. In ultrasonic diagnosis, the ultrasonic device is pressed against an object to be tested such as a human body. In pressing of the ultrasonic device, an ultrasonic gel sheet, for example, is interposed between the ultrasonic device and the object. The ultrasonic gel sheet is meant to achieve acoustic matching between the ultrasonic device and the object.

According to JP-A-2012-176197, an ultrasonic gel sheet has a primary echo gel film and a secondary echo gel film. The secondary echo gel film is directly placed on the primary echo gel film, and the secondary echo gel film is exposed to outside air. As the secondary echo gel film becomes dry, a dispersion medium, i.e., water moves from the primary echo gel film to the secondary echo gel film. Drying therefore proceeds, not only in the secondary echo gel film but also in the primary echo gel film.

In view of the above, an ultrasonic gel sheet assembly capable of preventing the loss of a dispersion medium as much as possible has been desired.

### SUMMARY

(1) According to an aspect of the invention, an ultrasonic gel sheet assembly used for acoustic matching between an ultrasonic device and an object to be tested, includes: a plurality of gel layers placed on top of each other; and a partition sheet placed between the plurality of gel layers to partition a dispersion medium contained in the gel layers between the gel layers.

Movement of water or any other dispersion medium is blocked by the partition sheet between the adjacent gel sheets placed on top of each other. Even if the dispersion medium is lost in one gel layer, the other gel layer is prevented from becoming dry. In this gel layer, the dispersion medium is sufficiently held. Therefore, even if the one gel layer loses the function of acoustic matching as it becomes dry, the other gel layer can sufficiently achieve the acoustic matching function. The gel layer moistened with the dispersion medium can be interposed between the ultrasonic device and the object to be tested. If the gel layers are directly placed on top of each other without the intervention of the partition sheet, as one gel layer becomes dry, the dispersion medium will move from the other gel layer to the one gel layer, resulting in that drying of the other gel layer will also proceed along with the drying of the one gel layer.

(2) The ultrasonic gel sheet assembly can further include coating sheets that cover outer surfaces of uppermost and lowermost gel layers out of the plurality of gel layers. The coating sheets protect the uppermost and lowermost gel layers from outside air. As long as being covered with the coating sheets, the uppermost and lowermost gel layers are prevented from loss of the dispersion medium. The gel layers can therefore be stored in a good state containing the dispersion medium after manufacture and also at a sale destination.

(3) The coating sheets may be formed of the same material as the partition sheet. With the partition sheet and the coating sheets being formed of the same material, the manufacture of the ultrasonic gel sheet assembly can be simplified. For example, when the uppermost and lowermost gel layers are peeled off, the partition sheet can function as the coating sheet as it is.

(4) Peripheries of the coating sheets may be sealed to enclose the plurality of gel layers therein. The coating sheets seal the multilayer body of the gel layers to prevent loss of the dispersion medium. The gel layers can therefore be stored in a good state containing the dispersion medium after manufacture and also at a sale destination. Since the coating sheets also serve as a packaging material, saving of the packaging material can be achieved.

(5) The coating sheets may be formed of a material different from that of the partition sheet. Because the coating sheets are peeled off at the use of the ultrasonic gel sheet assembly, it is unnecessary to consider reflection of ultrasound at the interfaces between the gel layers and the coating sheets. Therefore, a material suitable for the covering function of the coating sheets can be used without the necessity of considering the acoustic impedance of the coating sheets.

(6) The dispersion medium may be water, and the partition sheet may be formed of any of silicone rubber, natural rubber, polyethylene, polypropylene, polyethylene terephthalate, polycarbonate, paper, and cloth. Having such a combination of the dispersion medium and the partition sheet, reflection of ultrasound is prevented or reduced at the interfaces between the gel layers and the partition sheet. Therefore, ultrasound can be well transmitted from the ultrasonic device to the object to be tested.

(7) The partition sheet may have a contact angle with water of 70° to 140°. Having such a contact angle, the partition sheet is given a water repelling function, securing a nature of resisting the passage of water therethrough.

(8) The partition sheet may have a shape of projections and depressions formed by embossing. Having such a surface, the partition sheet can be easily peeled off from the gel layer.

(9) The partition sheet may have a tab extending outward from the outline of the gel layers. At the time of peeling off the partition sheet from the gel layer, the user can grip the tab. In this way, the partition sheet can be easily torn off from the gel layer.

### BRIEF DESCRIPTION OF THE DRAWINGS

The invention will be described with reference to the accompanying drawings, wherein like numbers reference like elements.

FIG. 1 is a partial perspective view schematically showing a structure of an ultrasonic gel sheet assembly according to the first embodiment of the invention.

FIG. 2 is a schematic view of one specific example of an electronic apparatus, i.e., an ultrasonic diagnosis apparatus according to an embodiment.

FIG. 3 is an enlarged front view of an ultrasonic probe.

FIG. 4 is a concept view schematically showing how to use an ultrasonic gel sheet assembly according to a specific example.

FIG. 5 is a concept view schematically showing how to use the ultrasonic gel sheet assembly in succession to FIG. 4.

FIG. 6 is an enlarged partial cross-sectional view of an ultrasonic gel sheet assembly schematically showing walls surrounding gel layers.

FIG. 7 is a concept view schematically showing how to use an ultrasonic gel sheet assembly according to another specific example.

FIG. 8 is a concept view schematically showing how to use the ultrasonic gel sheet assembly in succession to FIG. 7.

FIG. 9 is a partial perspective view schematically showing a structure of an ultrasonic gel sheet assembly according to the second embodiment of the invention.

FIG. 10 is a partial perspective view schematically showing a structure of an ultrasonic gel sheet assembly according to a variation of the second embodiment.

FIG. 11 is a partial perspective view schematically showing a structure of an ultrasonic gel sheet assembly according to the third embodiment of the invention.

### DESCRIPTION OF EXEMPLARY EMBODIMENTS

The following describe embodiments of the present invention with reference to the accompanying drawings. It is to be noted that the embodiments to be described hereinafter will not unduly limit the content of the invention defined in the appended claims and that all of configurations to be described in the embodiments are not necessarily essential for achievement of the invention.

(1) Configuration of Ultrasonic Gel Sheet Assembly of First Embodiment

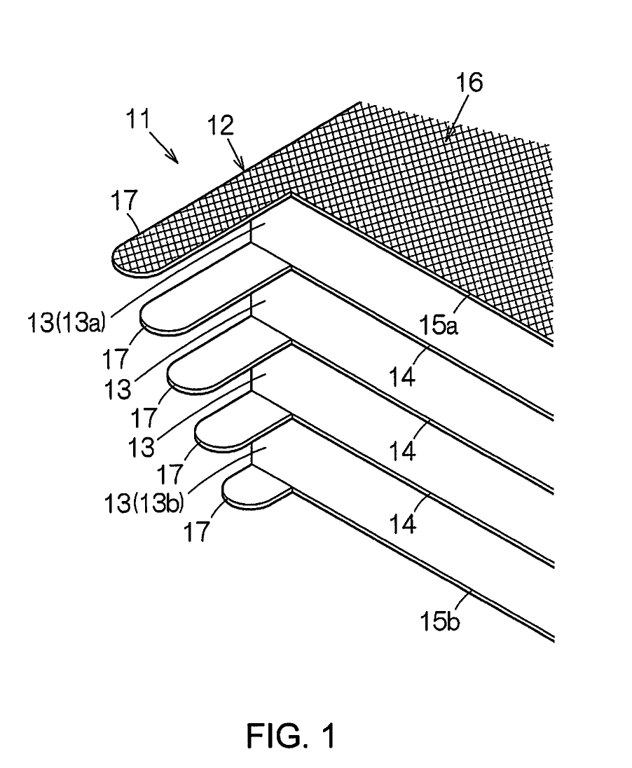

FIG. 1 schematically shows an ultrasonic gel sheet assembly 11 according to the first embodiment of the invention. The ultrasonic gel sheet assembly 11 has a multilayer body 12. The multilayer body 12 is in the shape of a sheet having a rectangular outline, for example, and has a plurality of gel layers 13 placed on top of each other. About 2 to 50 gel layers 13 are placed on top of each other in the multilayer body 12. A partition sheet 14 is placed between any adjacent gel layers 13. Coating sheets 15a and 15b are respectively placed on the outer surfaces of uppermost and lowermost gel layers 13a and 13b out of the gel layers 13. The coating sheets 15a and 15b cover the outer surfaces of the uppermost and lowermost gel layers 13a and 13b.

The gel layers 13 are formed of a gel material. The gel is formed by adding a thickening stabilizer (dispersoid) such as xanthan gum to water (dispersion medium). The gel maintains a fixed shape, for example, while having fluidity. The gel layers 13 are formed to have equal thicknesses: they have a thickness of about 0.05 mm to 5.0 mm, for example. The gel layers 13 may otherwise have thicknesses different from each other. Also, the thickness may vary in one gel layer 13.

The partition sheets 14 separate the dispersion medium contained in the gel layers 13 between the adjacent gel layers 13. The partition sheets 14 are formed of any of silicone rubber, natural rubber, polyethylene, polypropylene, polyethylene terephthalate, polycarbonate, paper, and cloth. The acoustic impedance of the partition sheets 14 is determined according to the acoustic impedance of the gel layers 13. Depending on the setting of the acoustic impedances, reflection of ultrasound is prevented or reduced as much as possible at the boundaries of the partition sheets 14 and the gel layers 13. In view of prevention of reflection of ultrasound, the acoustic impedance ratio of the partition sheets 14 to the gel layers 13 may be set in the range of 0.8 to 1.5.

The contact angle between the partition sheets 14/coating sheets 15a and 15b and water is 70° to 140° . Depending on this contact angle, the partition sheets 14 and the coating sheets 15a and 15b are given a water repelling function, securing a nature of resisting the passage of water therethrough. Moreover, the partition sheets 14 and the coating sheets 15a and 15b easily come off from the gel layers 13 compared with the skin of which the contact angle with water is 50° to 70°. In the illustrated example, the partition sheets 14 and the coating sheets 15a and 15b have surfaces in a shape of projections and depressions formed by embossing. The projections and depressions are formed in a mesh pattern 16. Such embossing can facilitate coming off of the partition sheets 14 and the coating sheets 15a and 15b from the gel layers 13.

In the illustrated example, the coating sheets 15a and 15b are formed of the same material as the partition sheets 14. The coating sheets 15a and 15b therefore isolate the dispersion medium contained in the uppermost and lowermost gel layers 13a and 13b from outside air. Evaporation of the dispersion medium from the outer surfaces of the uppermost and lowermost gel layers 13a and 13b is prevented. The coating sheets 15a and 15b cover the entire outer surfaces of the uppermost and lowermost gel layers 13a and 13b. The coating sheets 15a and 15b may otherwise be formed of a material different from that of the partition sheets 14.

As shown in FIG. 1, the partition sheets 14 and the coating sheets 15a and 15b each have a tab 17 extending outward from the outline of the gel layer 13. The tab 17 is formed to have a size large enough to allow the user to pinch it easily with his or her two fingers. The tab 17 is placed next to one corner of the rectangular outline, for example. With this placement, the force of tearing off with the tab 17 at one position can act on the entire sheet sufficiently. The tabs 17 may be formed so that the edges (tips) thereof be located farther from the outline of the gel layers 13 as the tabs 17 are closer to the uppermost one. Such tabs 17 can be pinched one by one in descending order from the uppermost one.

(2) Operation of Ultrasonic Gel Sheet Assembly (Part 1)

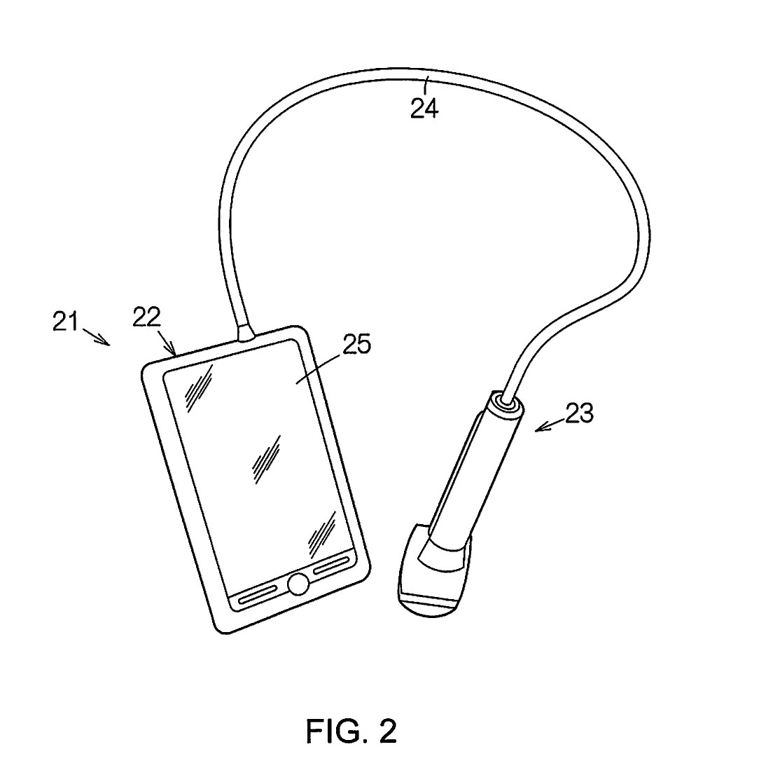

In formation of an ultrasonic image, an ultrasonic probe of an ultrasonic imaging apparatus such as an ultrasonic diagnosis apparatus is pressed against an object to be tested such as a human body, for example. In pressing of the ultrasonic probe, the ultrasonic gel sheet assembly 11 is interposed between the ultrasonic probe and the object. As shown in FIG. 2, an specific example of an ultrasonic diagnosis apparatus 21 includes a terminal 22 and an ultrasonic probe 23, for example. The terminal 22 and the ultrasonic probe 23 are connected to each other via a cable 24, and exchange electronic signals with each other via the cable 24. A display panel 25 is incorporated in the terminal 22. The screen of the display panel 25 is exposed at the surface of the terminal 22. In the terminal 22, an image is generated based on the ultrasound detected by the ultrasonic probe 23, and the imaged detected result is displayed on the screen of the display panel 25.

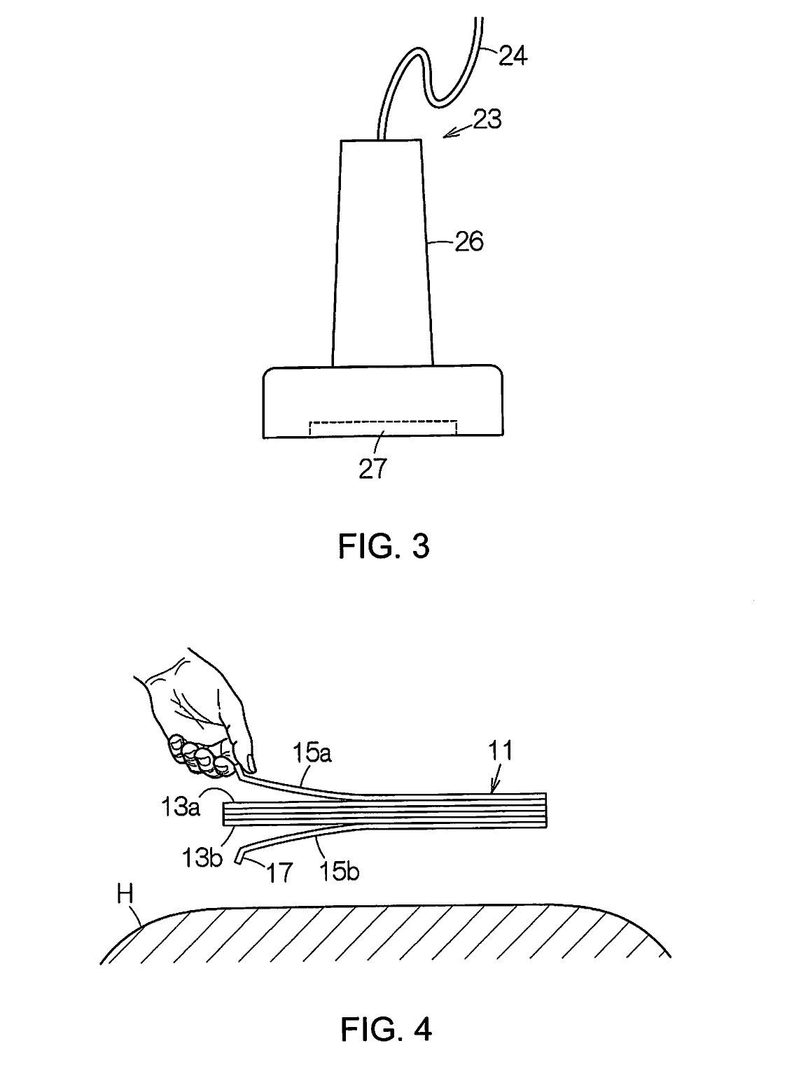

As shown in FIG. 3, the ultrasonic probe 23 has a housing 26. An ultrasonic device 27 is housed in the housing 26. The surface of the ultrasonic device 27 can be exposed at the surface of the housing 26. The ultrasonic device 27 outputs ultrasound from its surface and also receives a reflected wave of the ultrasound.

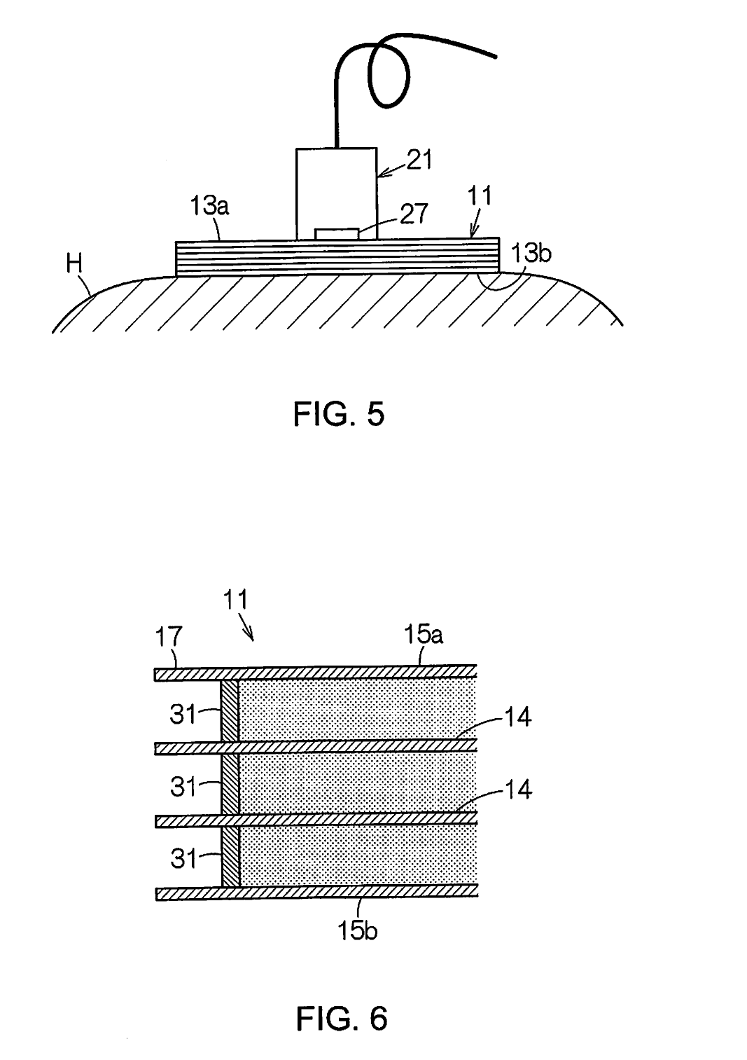

As shown in FIG. 4, at the use of the ultrasonic gel sheet assembly 11, the two coating sheets 15a and 15b are peeled off, whereby the outer surfaces of the uppermost and lowermost gel layers 13a and 13b are exposed. For peeling off, the tabs 17 of the coating sheets 15a and 15b may be pinched. As shown in FIG. 5, the outer surface of the lowermost gel layer 13b is stuck to an object H to be tested. Since the gel layer 13b is sufficiently moistened with the dispersion medium, the outer surface of the gel layer 13b comes into close contact with the object H. For example, pores of the skin are closed. Air is expelled from the interface. The ultrasonic device 27 is pressed against the outer surface of the uppermost gel layer 13a. Since the gel layer 13a is also sufficiently moistened with the dispersion medium, the ultrasonic device 27 can be brought into close contact with the outer surface of the gel layer 13a. Air is expelled from the interface. The ultrasonic device 27 can slide on the outer surface of the gel layer 13a. In this way, the ultrasonic device 27 and the object H are well coupled to each other acoustically. A good ultrasonic image can thus be formed.

When the object H is changed to another one, the partition sheet 14 adjacent to the lowermost gel layer 13b is peeled off from its adjacent gel layer 13. For peeling off, the tab 17 of the partition sheet 14 may be pinched. The lowermost gel layer 13b is removed together with the partition sheet 14. The gel layer 13 moistened with the dispersion medium is then newly exposed. The outer surface of the newly-exposed gel layer 13 is then stuck to an object H to be tested. Since the gel layer 13 is sufficiently moistened with the dispersion medium, the outer surface of the gel layer 13 comes into close contact with the object H. Similarly, every time the object H is changed to another one, the partition sheet 14 adjacent to the uppermost gel layer 13a may be peeled off from its adjacent gel layer 13. In this way, the uppermost gel layer 13a can be removed together with the partition sheet 14.

Otherwise, the partition sheet 14 adjacent to the uppermost gel layer 13a may be maintained over a plurality of objects H to be tested. In this case, the uppermost gel layer 13a is kept exposed to outside air. Therefore, the dispersion medium is gradually lost from the gel layer 13a. Once it is recognized that the uppermost gel layer 13a has become dry, the partition sheet 14 adjacent to the uppermost gel layer 13a may be peeled off from its adjacent gel layer 13. In this way, the uppermost gel layer 13a can be removed together with the partition sheet 14. The gel layer 13 moistened with the dispersion medium is then newly exposed.

Movement of water or any other dispersion medium is blocked between the gel layers 13 placed on top of each other. Even if the dispersion medium is lost from the gel layers 13a and 13b facing outside air, their adjacent gel layers 13 are prevented from drying: the dispersion medium is sufficiently held in the gel layers 13. Therefore, even if the function of acoustic matching is lost from the uppermost and lowermost gel layers 13a and 13b as they become dry, the other gel layers 13 can adequately achieve the function of acoustic matching. The gel layers 13 moistened with the dispersion medium can be interposed between the ultrasonic device 27 and the object H. If the gel layers 13 are directly placed on top of each other without the intervention of the partition sheet 14, as one gel layer 13 becomes dry, the dispersion medium will move from the other gel layer 13 to the one gel layer 13, resulting in that drying of the other gel layer 13 will also proceed along with the drying of the one gel layer 13.

The coating sheets 15a and 15b protect the uppermost and lowermost gel layers 13a and 13b from outside air. As long as being covered with the coating sheets 15a and 15b, the uppermost and lowermost gel layers 13a and 13b are prevented from losing the dispersion medium. The moist gel layers 13a and 13b can be well stored after manufacture and also at a sale destination. Moreover, since the partition sheets 14 and the coating sheets 15a and 15b are formed of the same material in the illustrated example, the manufacture of the ultrasonic gel sheet assembly 11 can be simplified. For example, when the uppermost and lowermost gel layers 13a and 13b are peeled off, the partition sheets 14 can function as the coating sheets 15a and 15b as they are. Otherwise, the coating sheets 15a and 15b may be formed of a material different from that of the partition sheets 14. Because the coating sheets 15a and 15b are peeled off at the use of the ultrasonic gel sheet assembly 11, it is unnecessary to consider reflection of ultrasound at the interfaces between the gel layers 13 and the coating sheets 15a and 15b. Therefore, no consideration on the acoustic impedance of the coating sheets 15a and 15b is necessary. A material suitable for the function of the coating sheets 15a and 15b can be used.

Water is used as the dispersion medium in the ultrasonic gel sheet assembly 11 according to this embodiment. In this case, the partition sheets 14 and the coating sheets 15a and 15b are formed of any of silicone rubber, natural rubber, polyethylene, polypropylene, polyethylene terephthalate, polycarbonate, paper, and cloth. Having such a combination of the dispersion medium and the partition sheets 14/coating sheets 15a and 15b, reflection of ultrasound is prevented or reduced at the interfaces between the gel layers 13 and the partition sheets 14/coating sheets 15a and 15b. Therefore, ultrasound can be well transmitted from the ultrasonic device 27 to the object H.

As described earlier, the contact angle between the surfaces of the the partition sheets 14/coating sheets 15a and 15b and water is 70° to 140°. With such a contact angle, the partition sheets 14 and the coating sheets 15a and 15b are given a water repelling function, securing a nature of resisting the passage of water therethrough. Moreover, the partition sheets 14 and the coating sheets 15a and 15b easily come off from the gel layers 13 compared with the skin of which the contact angle with water is 50° to 70°. In particular, since the mesh pattern 16 is formed on the surfaces of the partition sheets 14 and the coating sheets 15a and 15b by embossing, the partition sheets 14 and the coating sheets 15a and 15b can be easily peeled off from the gel layers 13. For example, the contact angle between a silicone rubber sheet and water (ultrapure water) was 113.3° to 114.1° (average: 113.7°) by the θ/2 method according to measurements using an automatic dynamic contact angle meter manufactured by Kyowa Interface Science Co., Ltd. Likewise, the contact angle between a polyethylene terephthalate (PET) film and water (ultrapure water) was 70.5° to 73.6° (average: 71.9°) by the θ/2 method according to measurements using the automatic dynamic contact angle meter manufactured by Kyowa Interface Science Co., Ltd.

In the ultrasonic gel sheet assembly 11, as shown in FIG. 6, a solid wall 31 may be placed along the outer rim of each gel layer 13. The wall 31 surrounds the gel layer 13 between the adjacent partition sheets 14 or between the partition sheet 14 and the coating sheet 15a or 15b along the surface of the partition sheet 14. The wall 31 may be formed by hardening the material of the gel layer 13 itself or formed of silicone rubber or any other solid material similar to that of the partition sheets 14. The wall 31 forms a storage space for the gel layer 13 in cooperation with the paired adjacent partition sheets 14 (one of which may be the coating sheet 15a or 15b). As a result, the dispersion medium can be prevented from evaporating from the outer rim of the gel layer 13, achieving further effective prevention of drying of the gel layer 13.

With the tab 17 extending outside from the outline of the gel layer 13 formed on each of the partition sheets 14 and the coating sheets 15a and 15b, the user can grip the tab 17 when peeling off the partition sheet 14 (coating sheet 15a or 15b) from the gel layer 13. In this way, the partition sheets 14 and the coating sheets 15a and 15b can be easily torn off from the gel layers 13. In particular, with the stacked partition sheets 14 and coating sheets 15a and 15b, the tabs 17 are avoided from completely overlapping one another but placed at displaced positions. Thus, the partition sheets 14 and the coating sheets 15a and 15b can be easily peeled off individually.

(3) Operation of Ultrasonic Gel Sheet Assembly (Part 2)

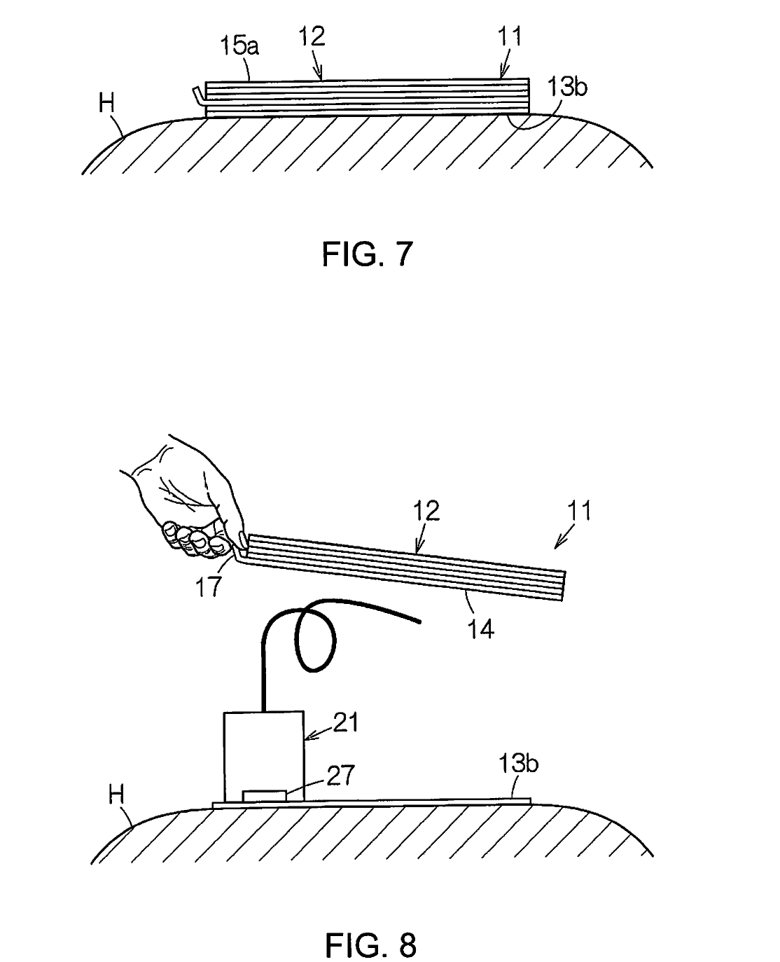

The coating sheet 15b covering the lowermost gel layer 13b is peeled off, whereby the outer surface of the lowermost gel layer 13b is exposed in the ultrasonic gel sheet assembly 11. For peeling off, the tab 17 of the coating sheet 15b may be pinched. At this time, the coating sheet 15a may maintain the covering of the uppermost gel layer 13a. As shown in FIG. 7, the outer surface of the lowermost gel layer 13b is stuck to an object H to be tested. Since the gel layer 13b is sufficiently moistened with the dispersion medium, the outer surface of the gel layer 13b comes into close contact with the object H. Air is expelled from the interface.

Subsequently, as shown in FIG. 8, the partition sheet 14 adjacent to the lowermost gel layer 13b that is in close contact with the object H is peeled off from the gel layer 13b. At this time, since the contact angle between the partition sheet 14 and water is larger than the contact angle between the skin and water, the partition sheet 14 comes off from the gel layer 13 more easily than the skin. Therefore, at the tearing off of the partition sheet 14, the gel layer 13 will not come off from the skin. The tab 17 of the partition sheet 14 may just be pinched at this peeling off. The multilayer body 12 including the layers located above this partition sheet 14 is separated together, and only the lowermost gel layer 13b remains on the object H. The ultrasonic device 27 is pressed against the surface of the remaining gel layer 13. Since the gel layer 13b is sufficiently moistened with the dispersion medium, the ultrasonic device 27 can be brought into close contact with the outer surface of the gel layer 13b. Air is expelled from the interface. The ultrasonic device 27 can slide on the outer surface of the gel layer 13b. In this way, the ultrasonic device 27 and the object H are well coupled to each other acoustically. A good ultrasonic image can thus be formed.

(4) Configuration of Ultrasonic Gel Sheet Assembly of Second Embodiment

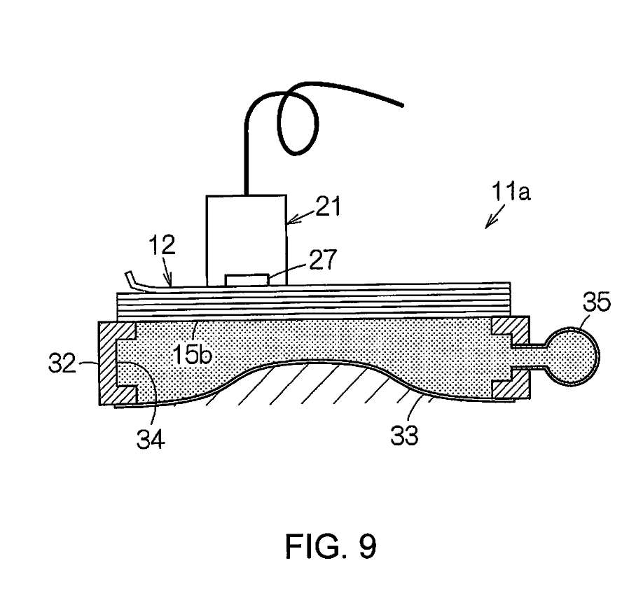

FIG. 9 schematically shows an ultrasonic gel sheet assembly 11a according to the second embodiment of the invention. The ultrasonic gel sheet assembly 11a has a multilayer body 12 as in the first embodiment. A frame 32 is secured to the coating sheet 15b of the multilayer body 12. The frame 32 may be formed of a metal material such as stainless steel and aluminum or a resin material such as polypropylene and polyethylene. The frame 32 continuously surrounds an inner space. The top end of the frame 32 supports the outer rim of the coating sheet 15b, and the coating sheet 15b adjoins the space inside the frame 32. The ratio of the acoustic impedance of the partition sheets 14 and the coating sheet 15b to the acoustic impedance of the gel layers 13 is set in the range of 0.8 to 1.5.

An acoustic matching film 33 is fixed to the bottom end of the frame 32. The acoustic matching film 33 is supported by the bottom end of the frame 32 along its outer rim, and adjoins the space inside the frame 32. Thus, an enclosed space 34 is formed by the acoustic matching film 33, the frame 32, and the coating sheet 15b. The enclosed space 34 in the frame 32 is filled with an acoustic matching medium such as water. The acoustic matching film 33 has flexibility, and can be deformed in response to the action of an external force. With such a deforming nature, the acoustic matching film 33 can follow the shape of the object H. The acoustic matching film 33 may be formed of a material having an acoustic impedance close to that of the object H. In the illustrated example, the acoustic matching film 33 is formed of natural rubber or silicone rubber.

A pressure regulation bag 35 is connected to the frame 32. The pressure regulation bag 35 is formed of a stretchable material. The inner space of the pressure regulation bag 35 is connected to the enclosed space 34 of the frame 32. When the volume of the enclosed space 34 changes with the deformation of the acoustic matching film 33, the volume of the pressure regulation bag 35 changes with this change. In this way, the total volume of these spaces is kept fixed. In the illustrated example, the Young's modulus of the multilayer body 12 is larger than that of the material of the pressure regulation bag 35.

In formation of an ultrasonic image, the ultrasonic gel sheet assembly 11a is pressed against the object H. The acoustic matching film 33 follows projections of the object H. At this time, according to the magnitude of the Young's modulus, while the multilayer body 12 maintains its original shape, the pressure regulation bag 35 expands. Thus, even if projections and depressions are present on the object H, the space between the object H and the ultrasonic gel sheet assembly 11a can be filled with an acoustic coupling material without causing deformation of the gel layers 13 and the partition sheets 14. The ultrasonic device 27 can therefore be coupled to the object H acoustically without fail.

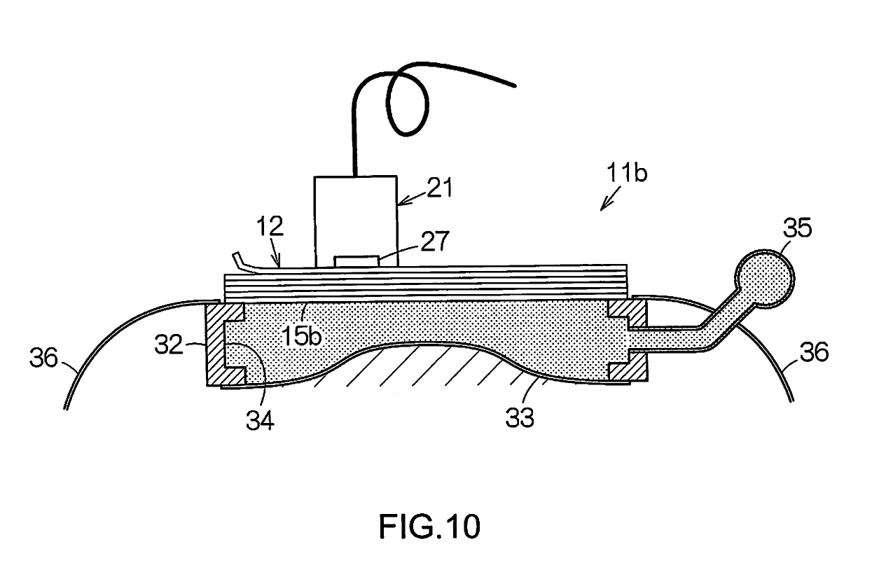

FIG. 10 schematically shows an ultrasonic gel sheet assembly 11b according to a variation of the second embodiment. In the ultrasonic gel sheet assembly 11b, the frame 32 is formed of an elastic material, and can follow the shape of the object H. Bands 36 are attached to the frame 32. The bands 36 can be formed of a stretchable material, for example. The ultrasonic gel sheet assembly 11b can be mounted on the object H, for example, with the bands 36. The bands 36 can be wound around, for example, an arm or leg of a human body. The other configuration is similar to that of the ultrasonic gel sheet assembly 11a.

(5) Configuration of Ultrasonic Gel Sheet Assembly of Third Embodiment

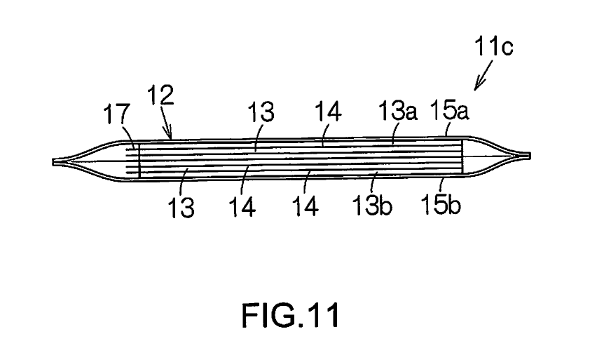

FIG. 11 schematically shows an ultrasonic gel sheet assembly 11c according to the third embodiment of the invention. The ultrasonic gel sheet assembly 11c has a multilayer body 12 as in the above embodiments. The multilayer body 12 is sealed in a packaging material 37. The packaging material 37 can be formed of a flexible resin sheet, for example. In the illustrated example, the packaging material 37 also serves as the coating sheets 15a and 15b of the multilayer body 12. In other words, the coating sheets 15a and 15b constitute part of the material the periphery of which is sealed to enclose the gel layers 13 therein. The other configuration is similar to those of the ultrasonic gel sheet assemblies 11, 11a, and 11b.

While the embodiments have been described in detail, it is to be easily understood by those skilled in the art that many modifications can be made without substantially departing from the novel matters and advantages of the invention. It is therefore construed that all of such modifications are included in the scope of the invention. For example, a term having appeared together with a broader or synonymous different term at least once in the description or any of the drawings can be replaced with such a different term at any position in the description or the drawing. It is also to be understood that the configurations and operations of the ultrasonic diagnosis apparatus 21, the ultrasonic probe 23, the ultrasonic device 27, etc. are not limited to those described in the embodiments, but can be modified in various ways.

The entire disclosure of Japanese Patent Application No. 2013-240698 filed on Nov. 21, 2013 is expressly incorporated by reference herein.

What is claimed is:

1. An ultrasonic gel sheet assembly used for acoustic matching between an ultrasonic device and an object to be tested, comprising:
a plurality of gel layers placed on top of each other; and
a partition sheet placed between the plurality of gel layers to partition a dispersion medium contained in the gel layers between the gel layers.

2. The ultrasonic gel sheet assembly of claim 1, further comprising coating sheets that cover outer surfaces of uppermost and lowermost gel layers out of the plurality of gel layers.

3. The ultrasonic gel sheet assembly of claim 2, wherein the coating sheets are formed of the same material as the partition sheet.

4. The ultrasonic gel sheet assembly of claim 2, wherein peripheries of the coating sheets are sealed to enclose the plurality of gel layers therein.

5. The ultrasonic gel sheet assembly of claim 2, wherein the coating sheets are formed of a material different from that of the partition sheet.

6. The ultrasonic gel sheet assembly of claim 1, wherein the dispersion medium is water, and the partition sheet is formed of any of silicone rubber, natural rubber, polyethylene, polypropylene, polyethylene terephthalate, polycarbonate, paper, and cloth.

7. The ultrasonic gel sheet assembly of claim 1, wherein the partition sheet has a contact angle with water of 70° to 140°.

8. The ultrasonic gel sheet assembly of claim 7, wherein the partition sheet has a shape of projections and depressions formed by embossing.

9. The ultrasonic gel sheet assembly of claim 1, wherein the partition sheet has a tab extending outward from an outline of the gel layers.
